(19) United States
(12) Patent Application Publication
(10) Pub. No.: US 2023/0194795 A1
(43) Pub. Date: Jun. 22, 2023

(54) Medical Ferrule Cleaning Device

(71) Applicant: Bard Access Systems, Inc., Salt Lake City, UT (US)

(72) Inventors: Steffan Sowards, Salt Lake City, UT (US); Anthony K. Misener, Bountiful, UT (US); William Robert McLaughlin, Bountiful, UT (US)

(21) Appl. No.: 18/086,497

(22) Filed: Dec. 21, 2022

Related U.S. Application Data
(60) Provisional application No. 63/293,020, filed on Dec. 22, 2021.

(51) Int. Cl. G02B 6/38; A61L 2/18

(52) U.S. Cl. CPC G02B 6/3866; A61L 2/18; B08B 2240/02

(57) ABSTRACT

An optical connector cleaning device configured to couple with and perform an optical cleaning and a disinfection of an optical connector of a medical device. The cleaning device includes a holding section and a cleaning section extending away from the holding section, the cleaning section extendable through a sterile barrier. A cleaning ribbon and a disinfecting ribbon, having a disinfectant absorbed therein, extend across a tip of the cleaning device so as to be sandwiched between the tip and the optical connector. An optical assessment system of the device evaluates optical transmission of the optical connector. Manipulation of actuators causes sliding displacement of the cleaning ribbon and the disinfecting ribbon across a tip of the cleaning device. Replaceable cartridges store unused and used portions of the cleaning ribbon and a disinfecting ribbon.

### PRIORITY

This application claims the benefit of priority to U.S. Provisional Application No. 63/293,020, filed Dec. 22, 2021, which is incorporated by reference in its entirety into this application.

### BACKGROUND

Fiber optic systems have been increasingly added to medical devices used in non-invasive and invasive medical procedures. Due to the fragile components of a fiber optic system, it can be difficult and time consuming to clean and decontaminate the entirety of the medical device before use. Proper cleaning of the fiber optic system is necessary to maintain proper fiber optic connection and extend the lifespan of the fiber optic system. Decontamination of the medical device is necessary to reduce contamination and extend the lifespan of the medical device. It would be beneficial to the user to have a cleaning apparatus configured to timely clean the fiber optic system and decontaminate the medical device.

Disclosed herein is a device and method that addresses the foregoing.

### SUMMARY

Disclosed herein is an optical connector cleaning device that, according to some embodiments, includes a device body having holding section and a cleaning section extending away from the holding section, where the cleaning section is configured to couple with an optical connector of a medical device. The device further includes an optical cleaning system configured for cleaning a fiber optic face of the optical connector, where the optical cleaning system includes a cleaning element disposed over a tip of the cleaning section, such that, when the cleaning section is coupled with the optical connector, the cleaning element is disposed between the tip and the fiber optic face.

In some embodiments, the cleaning element includes a ribbon, and cleaning a fiber optic face includes slidably displacing the cleaning element across the fiber optic face, and in some embodiments, the cleaning element extends along the cleaning section between the holding section and the tip. In some embodiments, during use, an unused portion of cleaning element is displaced along the cleaning section from the holding section to toward the tip, and a used portion of cleaning element is displaced along the cleaning section from the tip the holding section.

In some embodiments, the optical cleaning system includes a dispensing mechanism coupled with a dispensing actuator, and manipulating the dispensing actuator causes the cleaning element to slidably displace across the fiber optic face.

In some embodiments, the device is configured for use across a sterile barrier such that, during use, the cleaning section is disposed within a sterile environment, and the holding section is disposed outside the sterile environment.

In some embodiments, the device further includes a replaceable cleaning cartridge coupled with the holding section, where, during use, a quantity of the unused portion of the cleaning element and a quantity of the used portion of the cleaning element are stored within the replaceable cleaning cartridge.

In some embodiments, the cleaning section includes a latching member configured to secure the device to the optical connector, where securing the device to the optical connector maintains a clean state of the optical connector during non-use of the optical connector.

In some embodiments, the device further includes a disinfection system configured to the disinfect the optical connector, where the disinfection system includes a disinfecting element disposed over the tip of the cleaning section, such that, when the cleaning section is coupled with the optical connector, the disinfection element is disposed between the tip and the optical connector.

In some embodiments, the disinfecting element includes a ribbon, and disinfecting the optical connector includes slidably displacing the disinfecting element between the tip and the optical connector, and in some embodiments, the disinfecting element extends along the cleaning section between the holding section and the tip.

In some embodiments, during use, an unused portion of disinfecting element is displaced along the cleaning section from the holding section to toward the tip, and a used portion of disinfecting element is displaced along the cleaning section from the tip to the holding section.

In some embodiments, the disinfecting system includes a disinfecting mechanism coupled with a disinfecting actuator, and manipulating the disinfecting actuator causes the disinfecting element to slidably displace between the tip and the optical connector.

In some embodiments, the device further includes a replaceable disinfecting cartridge coupled with the holding section, where, during use, a quantity of the unused portion of the disinfecting element and a quantity of the used portion of the disinfecting element are stored within the replaceable cleaning cartridge.

In some embodiments, the disinfecting element includes a disinfectant absorbed therein, and slidably displacing the disinfecting element between the tip and the optical connector applies the disinfectant to the optical connector to disinfect the optical connector.

In some embodiments, the cleaning element and the disinfecting element are separate elements.

In some embodiments, the device further includes an optical assessment system configured to assess a capability of light transmission across the optical connector, and in some embodiments, the light transmission includes reflected light emanating from fiber optic Bragg gratings. In some embodiments, the optical assessment system is configured to assess the capability of light transmission when the cleaning section is coupled with the optical connector.

In some embodiments, the device further includes a cap configured for placement on the tip during non-use of the device. In some embodiments, the device further includes an adapter cap configured for attachment to the cleaning section, the adapter configured to enable coupling of the optical connector cleaning device to the optical connector.

Also disclosed herein is a method of cleaning an optical connector of a medical device that, according to some embodiments, includes (i) coupling a cleaning device to the optical connector, (ii) slidably displacing a cleaning ribbon of the cleaning device between the cleaning device and face of the fiber optic face of the optical connector, and transferring a disinfectant from the cleaning device to the optical connector.

In some embodiments of the method, transferring the disinfectant includes slidably displacing a disinfecting ribbon of the cleaning device between the cleaning device and the optical connector.

In some embodiments, the method further includes (i) inserting a cleaning section of the cleaning device through a sterile barrier into a sterile environment and (ii) maintaining a holding section of the cleaning device outside the sterile environment.

In some embodiments, the method further includes assessing a capability of light transmission across the optical connector when the cleaning device is coupled to the optical connector and in some embodiments of the method, the light transmission includes reflected light emanating from fiber optic Bragg gratings.

In some embodiments, the method further includes (i) securing the cleaning device to the optical connector and (ii) storing the medical device with the cleaning device secured to the optical connector.

These and other features of the concepts provided herein will become more apparent to those of skill in the art in view of the accompanying drawings and following description, which describe particular embodiments of such concepts in greater detail.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A illustrates a perspective view of an optical connector cleaning device, in accordance with some embodiments;

FIG. 1B is a detailed illustration of a tip portion of the cleaning device of FIG. 1A including a cleaning system, in accordance with some embodiments;

FIG. 1C is a detailed illustration of the tip portion of the cleaning device of FIG. 1A including a disinfecting system, in accordance with some embodiments;

FIG. 2A illustrates a cap for placement on a tip of the cleaning device of FIG. 1A, in accordance with some embodiments;

FIG. 2B illustrates an adapter for coupling of the cleaning device with the optical connector of FIG. 1A, in accordance with some embodiments;

FIG. 3 illustrates a perspective view of the cleaning device of FIG. 1A and a fiber optical system of depicting exemplary methods of cleaning optical connectors of the fiber optical system, in accordance with some embodiments; and

FIG. 4 illustrates a flow chart of an exemplary method of cleaning an optical connector, in accordance with some embodiments.

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The phrases “connected to,” “coupled to,” and “in communication with” refer to any form of interaction between two or more entities, including but not limited to mechanical, electrical, magnetic, electromagnetic, fluid, and thermal interaction. Two components may be coupled to each other even though they are not in direct contact with each other. For example, two components may be coupled to each other through an intermediate component.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Unless defined otherwise, all technical and scientific terms used herein have the same meaning as commonly understood by those of ordinary skill in the art.

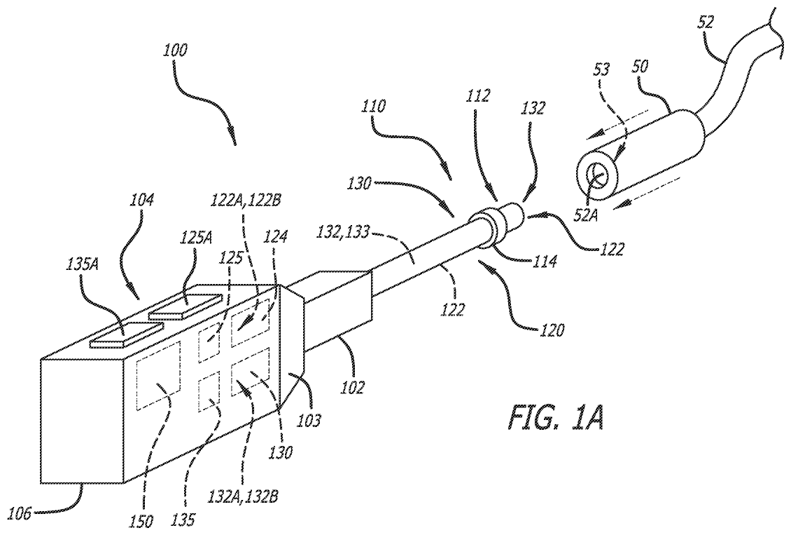

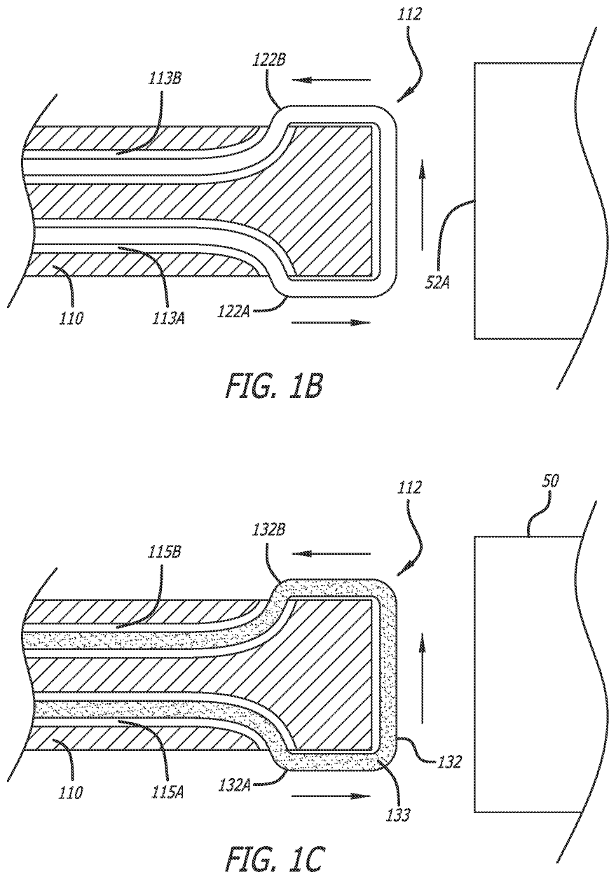

FIG. 1A illustrates a perspective view of optical connector cleaning device for cleaning an optical connector (e.g., a fiber optic ferrule) of a medical device in a medical setting, in accordance with some embodiments. The optical connector cleaning device (device) 100, is generally configured to replenish an optical connector, including a fiber optic ferrule, of a medical device after, during, and/or before use. In some instances, a face of an optical fiber, as disposed within the optical connector, may become contaminated, damaged, or otherwise rendered unsuitable for use. The device 100 is configured to repair or replenish the face of the optical fiber to enable continued use of the medical device.

In some instances, the optical connector may be located within a sterile environment during use. As such, microbial contamination of the optical connector may also render the optical connector, or the medical device as a whole, unsuitable for use in the instant case. Hence, disinfecting the optical connector may be necessary or advantageous so as to reduce a risk of infection to the patient. According to some embodiments, the device 100 may also be configured to disinfect the optical connector.

The device 100 includes a device body 102 having a holding section 104 configured for handling and manipulation by a user and a cleaning section 110 extending away from the holding section 104, where the cleaning section 110 is configured to couple with and operatively engage an optical connector 50. The holding section 104 may include a base 106 specifically configured for ergonomic handing by a user. In some embodiments, the device body 102 may include a tapered section 103 disposed between the holding section 104 and the cleaning section 110.

In some embodiments, the holding section 104 may be configured for use outside a sterile environment and the cleaning section 110 may be configured for use within the sterile environment. As such, in some embodiments, the device body 102 may be segmented to distinguish the cleaning section 110 from the holding section 104. In some instances, the sterile environment may be separated from a non-sterile environment by a sterile barrier, such as a drape, for example. Hence, in some embodiments, the device 100 may be configured for insertion of the cleaning section 110 across/through the sterile barrier from the non-sterile environment into the sterile environment, while the holding section 104 remains outside the sterile environment. The cleaning section 110 may generally define an elongate shape having a tip 112 disposed opposite the holding section 104.

In some embodiments, the cleaning section 110 may include a latching member 114, where the latching member is configured to enable securement of the device 100 to the optical connector 50. As such, the device 100, when secured to the optical connector 50, may provide protection to the optical connector 50 so as to prevent damage or contamination of the optical connector 50. In a similar fashion, the optical connector 50 may provide protection to the cleaning section 110, thereby protecting the tip 112 from damage or contamination.

The device 100 includes an optical cleaning system 120, where the optical cleaning system 120 is configured to restore the optical functionality of the optical connector 50. More specifically, the optical cleaning system 120 may be configured to replenish the optical functionality of a fiber optic face 52A of an optical fiber 52 or each face a number (e.g., 2, 3 4, 5 or more optical fibers) of the optical connector 50. In some embodiments, the optical connector 50 may include a number of electrical contacts 53. In such embodiments, the optical cleaning system 120 may be configured to clean or otherwise replenish the electrical contacts 53 and thereby, restore an electrical connecting functionality of the optical connector 50. It is noted that the optical connector 50 may be associated with any medical device utilizing fiber optic capability.

The optical cleaning system 120 may include a cleaning element 122 configured to contact the face 52A. More specifically, the cleaning element 122 may be configured to slide across the face 52A to clean, polish, or otherwise replenish the face 52A. The cleaning element 122 may include a ribbon or filament extending across the tip 112. The cleaning section 110 is configured to couple with the optical connector 50 such that the tip 112 abuts the face 52A sandwiching the cleaning element 122 therebetween. The tip 112 may exert a contact force on the face 52A to enhance a sliding engagement of the cleaning element 122 with the face 52A.

FIG. 1B is a cross-sectional view of a tip portion of the cleaning section 110 illustrating details of the optical cleaning system 120, according to some embodiments. The optical cleaning system 120 is generally configured to dispense the cleaning element 122 to the tip 112. In the illustrated embodiment, the optical cleaning system 120 is configured to dispense an unused portion 122A of the cleaning element 122 along the cleaning section 110 from the handling section 104 to the tip 112. In some embodiments, the optical cleaning system 120 may be configured to retrieve a used portion 122B of the cleaning element 122 along the cleaning section 110 from the tip 112 to the handling section 104. In some embodiments, retrieving the used portion 122B causes the cleaning element 122 to slide between the tip 112 and the face 52A. In some embodiments, a bulk or quantity of the unused portion 122A and the used portion 122B of the cleaning element 122 may be contained within the handling section 104. In some embodiments, the unused portion 122A may be disposed within a dispensing lumen 113A of the cleaning section 110 and the used portion 122B may be disposed within a retrieving lumen 113B of the cleaning section 110, where dispensing and retrieving lumens 113A, 113B extend along the cleaning section 110 between the tip 112 and the holding section 104.

Referring to FIGS. 1A and 1B, in some embodiments, the optical cleaning system 120 may include a cartridge system 124 disposed within the handling section 104 where the unused portion 122A is dispensed from the cartridge system 124 and the used portion 122B is retrieved by the cartridge system 124. In the illustrated embodiment, the cartridge system 124 (or the system 100 as a whole) includes a dispensing mechanism 125 coupled with an actuator 125A, where manipulating the actuator 125A may cause the cleaning element 122 to slide across the tip 112. An exemplary dispensing mechanism 125 may include a retrieving spool (not shown) having the used portion 122B wound thereon, and pressing the actuator 125A rotates the retrieving spool to draw the used portion 122B away from the tip 112. In some embodiments, the cleaning cartridge system 124 may be a consumable or disposable item, i.e., the cleaning cartridge system 124 may be replaceable within the holding section 104.

The device further 100 includes a disinfecting system 130 generally configured to disinfect the optical connector 50. The disinfecting system 130 is generally configured to apply a disinfectant 133 (i.e., a disinfecting solution) to the optical connector 50. More specifically, the disinfecting system 130 is configured to swab the optical connector 50 with the disinfectant 133. In some embodiments, the disinfecting system 130 may in some respects resemble the components and functionality of the optical cleaning system 120.

The disinfecting system 130 may include a disinfecting element 132 configured to disinfect the optical connector 50. More specifically, the disinfecting element 132 may be configured to contact the optical connector 50 including the face 52A and the electrical contacts 53, where contacting the optical connector 50 applies the disinfectant 133 thereto. The disinfecting element 132 may include a ribbon or filament extending across the tip 112, where the disinfecting element 132 contains the disinfectant 133. The cleaning section 110 is configured to engage the optical connector 50 such that the tip 112 engages the optical connector sandwiching the disinfecting element 132 therebetween. The tip 112 may exert a contact force on the optical connector 50 to enhance the application of the disinfectant 133 to the optical connector 50.

The disinfecting system 130 is generally configured to dispense the disinfecting element 132 to the tip 112. In the illustrated embodiment, the disinfecting system 130 is configured to dispense an unused portion 132A the disinfecting element 132 along the cleaning section 110 from the handling section 104 to the tip 112. In some embodiments, the disinfecting system 130 may be configured to retrieve a used portion 132B of the disinfecting element 132 along the cleaning section 110 from the tip 112 to the handling section 104. In some embodiments, retrieving the used portion 132B may cause the disinfecting element 132 to slide between the tip 112 and the optical connector 50. In some embodiments, a bulk of the unused portion 132A and the used portion 132B of the disinfecting element 132 may be contained within the handling section 104.

FIG. 1C is a cross-sectional view of a tip portion of the cleaning section 110 illustrating details of the disinfecting system 130, according to some embodiments. The disinfecting system 130 is generally configured to dispense the disinfecting element 132 to the tip 112. In the illustrated embodiment, the disinfecting system 130 is configured to dispense an unused portion 132A the disinfecting element 132 along the cleaning section 110 from the handling section 104 to the tip 112. In some embodiments, the disinfecting system 130 may be configured to retrieve a used portion 132B of the disinfecting element 132 along the cleaning section 110 from the tip 112 to the handling section 104. In some embodiments, retrieving the used portion 132B causes the disinfecting element 132 to slide between the tip 112 and the optical connector 50. In some embodiments, a bulk of the unused portion 132A and the used portion 132B of the disinfecting element 132 may be contained within the handling section 104. In some embodiments, the unused portion 132A may be disposed within a dispensing lumen 115A of the cleaning section 110 and the used portion 132B may be disposed within a retrieving lumen 115B of the cleaning section 110, where dispensing and retrieving lumens 115A, 115B extend along the cleaning section 110 between the tip 112 and the holding section 104.

The cleaning element 122 and the disinfecting element 132 may be disposed in various arrangements with respect to the tip 112. According to some embodiments, the cleaning element 122 may be disposed atop the disinfecting element 132. According to some embodiments, the cleaning element 122 may be disposed in parallel with the disinfecting element 132 across the tip 112. According to some embodiments, the disinfecting element 132 may travel across the tip 112 in the same direction as the cleaning element 122. According to some embodiments, the cleaning element 122 may be disposed perpendicular to the disinfecting element 132.

Referring to FIGS. 1A, 1C, in some embodiments, the disinfecting system 130 may include a disinfecting cartridge system 134 disposed within the handling section 104 where the unused portion 132A is dispensed from the disinfecting cartridge system 134 and the used portion 132B is retrieved by the disinfecting cartridge system 134. In the illustrated embodiment, the disinfecting cartridge system 134 (or the system 100 as a whole) includes a dispensing mechanism 135 coupled with a disinfecting actuator 135A, where manipulating the disinfecting actuator 135A causes the disinfecting element 132 to slide across the tip 112. An exemplary dispensing mechanism 135 may include a retrieving spool (not shown) having the used portion 132B wound thereon, and pressing the disinfecting actuator 135A rotates the retrieving spool to draw the used portion 132B away from the tip 112, thereby disposing a segment of the unused portion 132A over the tip 112. Such an exemplary dispensing mechanism 135 may also include a solution container (not shown) having the disinfectant 133 and the unused portion 132A stored therein. In some embodiments, disinfecting cartridge system 134 may be a consumable or disposable item, i.e., the disinfecting cartridge system 134 may be replaceable within the holding section 104.

In some embodiments, the disinfecting system 130 may be combined with the optical cleaning system 120. According to one example, a number of components of the cleaning system 120 may be shared with the disinfecting system 130. According to another example, disinfecting system 130 may be incorporated into the optical cleaning system 120. In some embodiments, the disinfecting element 132 may be incorporated into the cleaning element 122, e.g., the cleaning element 122 or portions thereof may include the disinfectant 133.

According to still another embodiment, the disinfecting system 130 may be configured to dispense the disinfectant 133 to the tip 112 via a fluid lumen (not shown) extending along the cleaning section 110. In such an embodiment, manipulating the disinfecting actuator 135A delivers the disinfection solution 133 to the cleaning element 122 at the tip 112 via the fluid lumen, where the cleaning element 122 applies the disinfection solution 133 to the optical connector 50.

According to another embodiment, the disinfecting element 132 includes separate pads, where the pads are individually attachable to the tip 112. During use, the user may physically couple the pad to the tip 112 so that engaging the tip 112 with the optical connector 50 applies the disinfection solution 133 to the optical connector 50.

Referring to the FIG. 1A, the cleaning section 110 may include a fiber optic assessment system 150, according to some embodiments. The fiber optic assessment system 150 may be configured to assess (or test) the capability of light transmission across the face 52A of the optical fiber 52 or the optical functionality of the optical connector 50 or the optical fiber 52 generally. In some embodiments, the fiber optic assessment system 150 may be configured to assess the light reflection capability of the optical fiber 52 as may be defined by Bragg gratings of the optical fiber 52. In some embodiments, the fiber optic assessment system 150 may include a portion of an optical loss test set or a portion of an optical time domain reflectometer. In some embodiments, the fiber optic assessment system 150 may be activated when the optical connector 50 is coupled with the tip 112. In some embodiments, the fiber optic assessment system 150 may include a console, an energy source, a light source, an optical receiver, one or more optical fibers, non-transitory computer readable medium, and a plurality of logic modules configured to assess the capability of light transmission and/or of the fiber light reflection of the optical fiber 52. In some embodiments, the fiber optic assessment system 150 may be in wireless communication with a computing device and communicate the capability and light reflection of the optical connector 50, thereto. In some embodiments, the fiber optic assessment system 150 may include a notification device, such as an illumination device, for example, to indicate a result of the assessment.

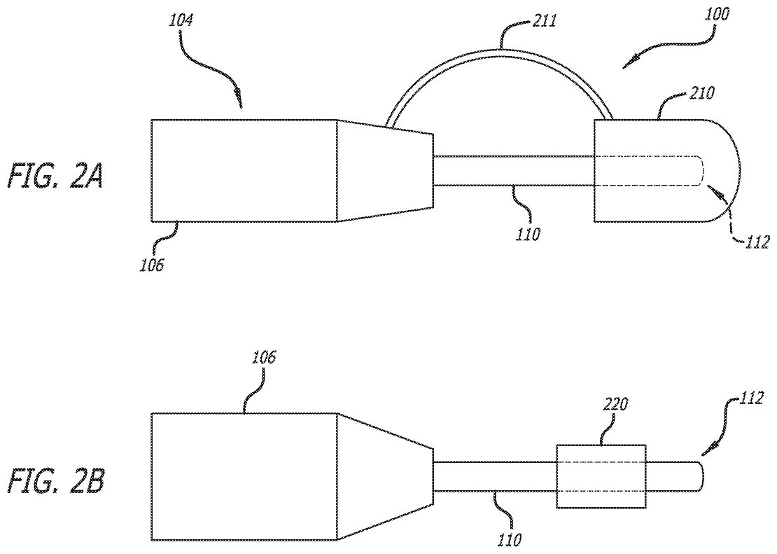

With reference to FIG. 2A, in some embodiments, the device 100 may include a cap 210 configured to couple with the cleaning section 110 and cover the tip 112. In some embodiments, the device 100 may include a tether 211 extending between the cap 210 and the device body 106, e.g., the holding section 104. In some embodiments, the cleaning element 122 and/or the disinfecting element 132 may be prone to damage. As such, the cap 210 may be configured to protect the cleaning element 122, the disinfecting element 132, and/or the tip 112 generally during non-use of the device 100. The cap 210 may also be configured to inhibit evaporation of the disinfectant 133 from the disinfecting element 132.

With reference to FIG. 2B, in some embodiments, the device 100 may include a number (e.g., 1, 2, 3 or more) of adapters 220 attachable to the cleaning section 110. The adaptors 220 may enable the device 100 (e.g., the cleaning section 110) to couple with different configurations of optical connectors 50. During use, the user may choose an adapter 220 consistent with an instant optical connector 50 and attach the chosen adapter 220 to the cleaning section 110. The user may then couple and/or secure the device 100 to the optical connector 50 via the chosen adapter 220.

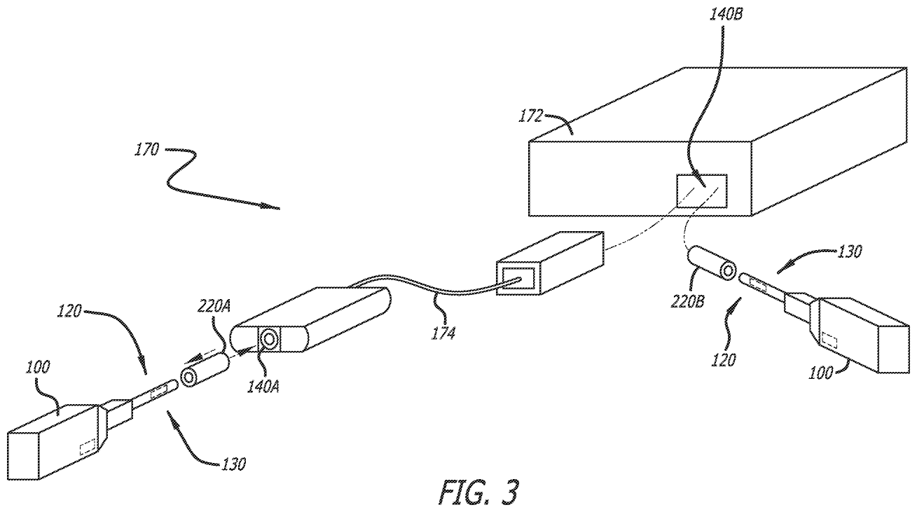

FIG. 3 illustrates a perspective view of the cleaning device 100 in accordance with an exemplary method of cleaning one or more optical connectors 140 of a fiber optic system 170 using the device 100. In some embodiments, the fiber optic system 170 may include an optical interrogator 172 configured to couple with patch cable 174 having an optical connector 140A. In some embodiments, the device 100 may be used to clean an optical connector 140B of the optical interrogator 172. In some embodiments, a first adapter 220A may be coupled to the device 100 to enable coupling of the device 100 with the optical connector 140A of the patch cable 174. In some embodiments, a second adapter 220B may be coupled with the device 100 to allow the device 100 to couple with the optical connector 140B of the optical interrogator 172.

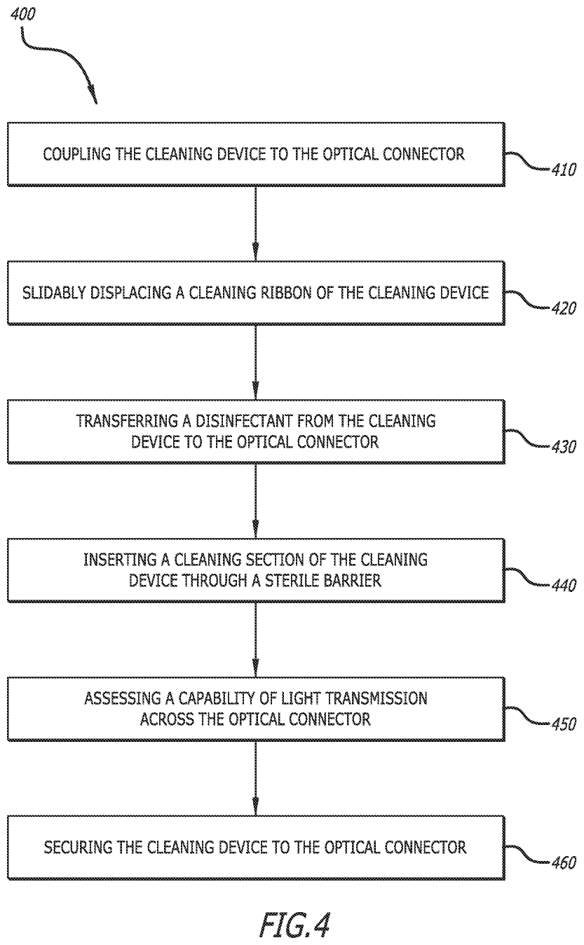

FIG. 4 illustrates a flow chart of an exemplary method of cleaning an optical connector 140, in accordance with some embodiments. The method 400 may include all or any subset of the following steps, actions, or processes. The method 400 may include coupling the cleaning device to the optical connector (block 410).

The method 400 may further include slidably displacing a cleaning ribbon of the cleaning device (block 420) where slidably displacing a cleaning ribbon takes place between the cleaning device and a fiber optic face of the optical connector.

The method 400 may include transferring a disinfectant from the cleaning device to the optical connector (block 430). In some embodiments of the method 400, transferring the disinfectant includes slidably displacing a disinfecting ribbon of the cleaning device between the cleaning device and the optical connector.

The method 400 may further include inserting a cleaning section of the cleaning device through a sterile barrier (block 440) into a sterile environment while maintaining the holding section of the cleaning device outside the sterile environment.

The method 400 may further include assessing a capability of light transmission across the optical connector (block 450). In some embodiments, the assessing a capability of light transmission may occur when the cleaning device is coupled to the optical connector. In some embodiments of the method 400, the light transmission includes assessing reflected light emanating from fiber optic Bragg gratings.

The method 400 may further include securing the cleaning device to the optical connector (block 460), and the method 400 may further include storing the medical device with the cleaning device secured to the optical connector.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. An optical connector cleaning device, comprising:
a device body including holding section and a cleaning section extending away from the holding section, the cleaning section configured to couple with an optical connector of a medical device; and
an optical cleaning system configured for cleaning a fiber optic face of the optical connector, the optical cleaning system including a cleaning element disposed over a tip of the cleaning section, such that, when the cleaning section is coupled with the optical connector, the cleaning element is disposed between the tip and the fiber optic face.

2. The device according to claim 1, wherein:
the cleaning element includes a ribbon, and
cleaning a fiber optic face includes slidably displacing the cleaning element across the fiber optic face.

3. The device according to claim 2, wherein the cleaning element extends along the cleaning section between the holding section and the tip.

4. The device according to claim 3, wherein during use:
an unused portion of cleaning element is displaced along the cleaning section from the holding section to toward the tip, and
a used portion of cleaning element is displaced along the cleaning section from the tip to the holding section.

5. The device according to claim 4, wherein:
the optical cleaning system includes a dispensing mechanism coupled with a dispensing actuator, and
manipulating the dispensing actuator causes the cleaning element to slidably displace across the fiber optic face.

6. The device according to claim 4, further comprising:
a replaceable cleaning cartridge coupled with the holding section,
wherein, during use, a quantity of the unused portion of the cleaning element and a quantity of the used portion of the cleaning element are stored within the replaceable cleaning cartridge.

7. The device according to claim 1, wherein the device is configured for use across a sterile barrier such that, during use:
the cleaning section is disposed within a sterile environment, and
the holding section is disposed outside the sterile environment.

8. The device according to claim 1, wherein the cleaning section includes a latching member configured to secure the device to the optical connector to maintain a clean state of the optical connector during non-use of the optical connector.

9. The device according to claim 1, further comprising:
a disinfection system configured to the disinfect the optical connector, the disinfection system including a disinfecting element disposed over the tip of the cleaning section, such that, when the cleaning section is coupled with the optical connector, the disinfection element is disposed between the tip and the optical connector.

10. The device according to claim 9, wherein:
the disinfecting element includes a ribbon, and
disinfecting the optical connector includes slidably displacing the disinfecting element between the tip and the optical connector.

11. The device according to claim 10, wherein the disinfecting element extends along the cleaning section between the holding section and the tip.

12. The device according to claim 11, wherein during use:
an unused portion of disinfecting element is displaced along the cleaning section from the holding section to toward the tip, and
a used portion of disinfecting element is displaced along the cleaning section from the tip to the holding section.

13. The device according to claim 12, wherein:
the disinfecting system includes a disinfecting mechanism coupled with a disinfecting actuator, and
manipulating the disinfecting actuator causes the disinfecting element to slidably displace between the tip and the optical connector.

14. The device according to claim 12, further comprising:
a replaceable disinfecting cartridge coupled with the holding section,
wherein, during use, a quantity of the unused portion of the disinfecting element and a quantity of the used portion of the disinfecting element are stored within the replaceable cleaning cartridge.

15. The device according to claim 10, wherein:
the disinfecting element includes a disinfectant absorbed therein, and
slidably displacing the disinfecting element between the tip and the optical connector applies the disinfectant to the optical connector to disinfect the optical connector.

16. The device according to claim 9, wherein the cleaning element and the disinfecting element are separate elements.

17. The device according to claim 1, further comprising:
an optical assessment system configured to assess a capability of light transmission across the optical connector.

18. The device according to claim 17, wherein the light transmission includes reflected light emanating from fiber optic Bragg gratings.

19. The device according to claim 17, wherein the optical assessment system is configured to assess the capability of light transmission when the cleaning section is coupled with the optical connector.

20. The device according to claim 1, further comprising:
a cap configured for placement on the tip during non-use of the device,
wherein the cap is configured to cover the tip.

21. The device according to claim 1, further comprising:
an adapter cap configured for attachment to the cleaning section, the adapter configured to enable coupling of the optical connector cleaning device to the optical connector.

22. A method of cleaning an optical connector of a medical device, comprising:
coupling a cleaning device to the optical connector;
slidably displacing a cleaning ribbon of the cleaning device between the cleaning device and face of the fiber optic face of the optical connector; and
transferring a disinfectant from the cleaning device to the optical connector.

23. The method according to claim 22, wherein transferring the disinfectant includes slidably displacing a disinfecting ribbon of the cleaning device between the cleaning device and the optical connector.

24. The method according to claim 22, wherein coupling the cleaning device includes:
inserting a cleaning section of the cleaning device through a sterile barrier into a sterile environment; and
maintaining a holding section of the cleaning device outside the sterile environment.

25. The method according to claim 22, further comprising:
assessing a capability of light transmission across the optical connector when the cleaning device is coupled to the optical connector.

26. The method according to claim 25, wherein the light transmission includes reflected light emanating from fiber optic Bragg gratings.

27. The method according to claim 22, further comprising:
securing the cleaning device to the optical connector; and
storing the medical device with the cleaning device secured to the optical connector.
